(19) United States
(12) Patent Application Publication
(10) Pub. No.: US 2025/0243192 A1
(43) Pub. Date: Jul. 31, 2025

(54) CRYSTALLINE FORM OF LANIFIBRANOR

(71) Applicant: INVENTIVA, Daix (FR)

(72) Inventors: Frédéric BELL, Messigny-Et-Vantoux (FR); Benaissa BOUBIA, Saint Apollinaire (FR)

(21) Appl. No.: 18/854,287

(22) PCT Filed: Apr. 4, 2023
(86) PCT No.: PCT/EP2023/058743

§ 371 Date: Oct. 4, 2024

(30) Foreign Application Priority Data
Apr. 5, 2022 (EP) 22305456.0

(51) Int. Cl. C07D 417/12; A61K 31/428

(52) U.S. Cl. CPC C07D 417/12; A61K 31/428

(57) ABSTRACT

The present invention relates to a crystalline form of lanifibranor having an X-ray diffraction pattern in accordance with FIG. 1.

### FIELD OF THE INVENTION

The present invention relates to a crystalline form of lanifibranor, to pharmaceutical compositions containing said crystalline form, and to the use of said crystalline form in therapy.

### BACKGROUND OF THE INVENTION

Lanifibranor or 1-(6-benzothiazolylsulfonyl)-5-chloro-1H-indole-2-butanoic acid is a pan-PPAR agonist which is currently in clinical development for the treatment of patients with non-alcoholic steatohepatitis (NASH), for which there is currently no approved therapy.

Lanifibranor is described as the free base in example 117 of WO 2007/026097, where it is obtained as a pale yellow powder having a melting point of 74-80° C. Crystalline forms of lanifibranor are disclosed in WO 2022/122014, WO 2022/143479, WO 2022/258060, WO 2022/261410 and WO 2023/016319.

The present invention provides a crystalline form of lanifibranor having desirable properties, such as high crystallinity, high purity, low hygroscopicity, favourable mechanical properties, and/or favourable stability.

### SUMMARY OF THE INVENTION

In one aspect, the invention provides a crystalline form of lanifibranor (form beta). Form beta of lanifibranor is characterized by one or more of the following methods: (1) powder X-ray diffraction (PXRD); (2) differential scanning calorimetry (DSC); (3) thermogravimetry (TGA); (4) dynamic vapor sorption (DVS); (5) infrared spectroscopy (IR).

In another aspect, the invention provides a method for preparing crystalline form beta of lanifibranor, the method comprising heating a solution of lanifibranor in acetic acid and slowly cooling the resulting solution to room temperature.

In another aspect, the invention provides a pharmaceutical composition comprising crystalline form beta of lanifibranor and a pharmaceutically acceptable carrier or excipient. In another aspect the invention provides a method of treating non-alcoholic fatty liver disease, which comprises administering to a subject in need thereof an effective amount of crystalline form beta of lanifibranor.

In another aspect the invention provides a method of treating a cirrhotic subject at risk of progressing from compensated stage to decompensated stage, which comprises administering to the subject an effective amount of crystalline form beta of lanifibranor. In another aspect the invention provides crystalline form beta of lanifibranor for use in a method of treating non-alcoholic fatty liver disease.

In another aspect the invention provides crystalline form beta of lanifibranor for use in a method of treating a cirrhotic subject at risk of progressing from compensated stage to decompensated stage.

### DESCRIPTION OF THE FIGURES

FIG. 1 shows the PXRD pattern of form beta of lanifibranor.

FIG. 2 shows the DSC curve of form beta of lanifibranor.

FIG. 3 shows the TGA curve of form beta of lanifibranor.

FIG. 4 shows the DVS isotherm plot of form beta of lanifibranor.

FIGS. 5A and 5B shows the IR spectrum of form beta of lanifibranor and the indexation of the absorption bands for the IR analysis.

FIG. 6 shows a comparison of PXRD patterns of amorphous form (bottom curve), form beta (middle curve), and form alpha (top curve) of lanifibranor.

FIG. 7 shows the DSC curve of form alpha of lanifibranor.

FIG. 8 shows a comparison of DSC curves of amorphous form (bottom curve), form beta (middle curve), and form alpha (top curve) of lanifibranor.

FIGS. 9-14 show the PXRD patterns of a suspension of a mixture of lanifibranor forms alpha and beta in various solvents: acetone (FIG. 9), ethanol (FIG. 10), ethyl acetate (FIG. 11), acetic acid (FIG. 12), methyl ethyl ketone (FIG. 13) and methyl isobutyl ketone (FIG. 14). In each figure, the top curve is the PXRD pattern of lanifibranor form alpha; the top/middle curve is the PXRD pattern of the suspension at T0; the middle/bottom curve is the PXRD pattern of the suspension at T0+24 h; the bottom curve is the PXRD pattern of lanifibranor form beta.

FIG. 15 shows the PXRD pattern of lanifibranor form alpha before (top curve) and after (bottom curve) compression.

FIGS. 16-17 show a superimposition of the PXRD pattern of the solid form CSI described in WO 2022/122014 (bottom curve) with the PXRD pattern of lanifibranor form beta as shown in FIG. 1 (top curve).

FIGS. 18-19 show a superimposition of the PXRD pattern of the solid form CSII described in WO 2022/122014 (bottom curve) with the PXRD pattern of lanifibranor form beta as shown in FIG. 1 (top curve).

FIG. 20 shows a superimposition of the PXRD pattern of the solid form CSIV described in WO 2022/122014 (bottom curve) with the PXRD pattern of lanifibranor form beta as shown in FIG. 1 (top curve).

FIG. 21 shows a superimposition of the PXRD patterns of the solid forms described in WO 2022/143479 with the PXRD pattern of lanifibranor form beta as shown in FIG. 1. The following forms are represented, from top to bottom: lanifibranor form beta; lanifibranor cinnamamide co-crystal; lanifibranor p-toluenesulfonic acid co-crystal; lanifibranor tromethamine salt; lanifibranor form A.

FIG. 22 shows a superimposition of the PXRD pattern of form A described in WO 2022/143479 (bottom curve) with the PXRD pattern of lanifibranor form alpha as shown in FIG. 1 (top curve).

FIG. 23 shows a superimposition of the PXRD patterns of solid forms CM-A, CM-B and CM-F described in WO 2022/258060 with the PXRD pattern of lanifibranor form beta as shown in FIG. 1. The following forms are represented, from top to bottom: lanifibranor form beta; form CM-A; form CM-B; form CM-F.

FIG. 24 shows a superimposition of the PXRD patterns of solid forms CM-C, CM-D, CM-E, CM-G and CM-I described in WO 2022/258060 with the PXRD pattern of lanifibranor form beta as shown in FIG. 1. The following forms are represented, from top to bottom: lanifibranor form beta; form CM-C; form CM-D; form CM-E; form CM-G; form CM-I.

FIG. 25 shows a superimposition of the PXRD patterns of solid forms LN-1, LN-2, LN-3 and LN-4 described in WO 2022/261410 with the PXRD pattern of lanifibranor form beta as shown in FIG. 1. The following forms are represented, from top to bottom: lanifibranor form beta; form LN-1; form LN-2; form LN-3; form LN-4.

FIG. 26 shows a superimposition of the PXRD pattern of form LN-1 described in WO 2022/261410 (bottom curve) with the PXRD pattern of lanifibranor form alpha as shown in FIG. 1 (top curve).

FIG. 27 shows a superimposition of the PXRD patterns of solid forms CSV (embodiments 2 and 3) and CSIII described in WO 2023/016319 with the PXRD pattern of lanifibranor form beta as shown in FIG. 1. The following forms are represented, from top to bottom: lanifibranor form beta; form CSV (embodiment 2); form CSV (embodiment 3); form CSIII. In all figures showing a PXRD pattern, the abscissa represents 2Theta) (° and the ordinate represents intensity (counts).

### DETAILED DESCRIPTION OF THE INVENTION

As used herein, the term “lanifibranor” is understood to mean lanifibranor free acid, i.e. the compound of formula:

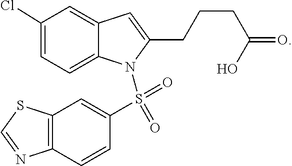 [SMILES: O=C(O)CCCc1cc2cc(Cl)ccc2n1S(=O)(=O)c1ccc2ncsc2c1]

In one aspect, the invention provides a crystalline form of lanifibranor (form beta). As described herein, lanifibranor (form beta) was characterized by one or more of the following methods: (1) powder X-ray diffraction (PXRD); (2) differential scanning calorimetry (DSC); (3) thermogravimetry (TGA). Such crystalline form may be further characterized by additional techniques, such as: (4) dynamic vapor sorption (DVS); and (5) infrared spectroscopy (IR).

In some embodiments of each aspect of the invention, form beta of lanifibranor is characterized by its PXRD pattern. One skilled in the art will appreciate that, with respect to X-ray diffraction peak positions (20), said positions will show some variability, typically as much as ±0.2°, such as for example ±0.1°. Further, one skilled in the art will appreciate that relative peak intensities will show inter-apparatus variability, as well as variability due to the degree of crystallinity, and should be taken as qualitative measures only.

In other embodiments of each aspects of the invention, lanifibranor (form beta) is characterized by its DSC curve. In still other embodiments of each aspects of the invention, lanifibranor (form beta) is characterized by its TGA curve.

It will be understood that various combinations of two, three or four techniques may be used to uniquely characterize lanifibranor (form beta) disclosed herein.

In one embodiment, lanifibranor (form beta) has a PXRD pattern comprising one, two, three, four, five or more than five peaks selected from the peaks in Table 1 (expressed in° 20+) 0.2°.

In one embodiment, lanifibranor (form beta) has a PXRD pattern comprising one or more peaks at 2θ values selected from 16.0°+0.2°, 18.7°+0.2° and 24.6°+0.2° 2θ, as measured using an X-ray wavelength of 1.5406 Å. In some embodiments, form beta has a PXRD pattern further comprising at least one peak at 2θ values selected from 11.4°+0.2°, 18.0+0.2°, 21.2+0.2°, 22.8+0.2°, 23.5+0.2°, 26.1+0.2° and 26.7°+0.2° 2θ, as measured using an X-ray wavelength of 1.5406 Å.

In some of such embodiments, the PXRD pattern further comprises one or more additional peaks at 20 values selected from the peaks in Table 1.

TABLE 1

| 2θ position | Relative |
|---|---|
| (°) | intensity |
| 9.3 | 4.6% |
| 10.5 | 2.5% |
| 11.1 | 21.2% |
| 11.4 | 60.4% |
| 12.0 | 40.0% |
| 12.6 | 5.7% |
| 13.2 | 5.3% |
| 13.4 | 2.8% |
| 14.3 | 24.2% |
| 15.0 | 21.4% |
| 16.0 | 100.0% |
| 16.4 | 6.5% |
| 18.0 | 55.4% |
| 18.3 | 41.4% |
| 18.7 | 91.5% |
| 18.9 | 20.0% |
| 19.9 | 39.1% |
| 20.2 | 42.3% |
| 20.6 | 36.8% |
| 21.2 | 47.4% |
| 22.1 | 12.2% |
| 22.2 | 10.4% |
| 22.5 | 7.8% |
| 22.8 | 77.1% |
| 23.3 | 3.9% |
| 23.5 | 50.9% |
| 23.9 | 8.3% |
| 24.0 | 31.2% |
| 24.6 | 91.7% |
| 24.8 | 39.5% |
| 25.1 | 3.8% |
| 25.2 | 7.8% |
| 25.5 | 7.9% |
| 26.1 | 46.6% |
| 26.7 | 64.6% |
| 27.3 | 18.8% |
| 27.7 | 2.2% |
| 28.2 | 20.5% |
| 28.6 | 3.3% |
| 28.7 | 1.7% |
| 29.0 | 1.0% |
| 29.2 | 3.0% |
| 29.4 | 3.1% |
| 30.0 | 23.2% |
| 30.3 | 9.6% |
| 30.5 | 8.1% |
| 30.7 | 5.5% |
| 31.7 | 4.5% |
| 32.1 | 8.9% |
| 32.3 | 13.6% |
| 32.7 | 5.5% |
| 32.9 | 9.1% |
| 33.2 | 14.0% |
| 33.3 | 9.9% |
| 33.6 | 1.8% |
| 34.4 | 4.3% |
| 34.5 | 3.4% |
| 34.8 | 8.0% |
| 35.0 | 9.6% |
| 35.4 | 1.5% |
| 36.2 | 14.6% |
| 36.3 | 11.2% |
| 36.5 | 1.5% |
| 37.0 | 10.0% |
| 37.2 | 7.3% |
| 38.0 | 1.7% |
| 38.1 | 1.0% |
| 38.4 | 3.9% |
| 38.5 | 8.7% |
| 38.6 | 5.2% |
| 39.1 | 1.5% |
| 39.4 | 1.8% |
| 39.8 | 4.3% |

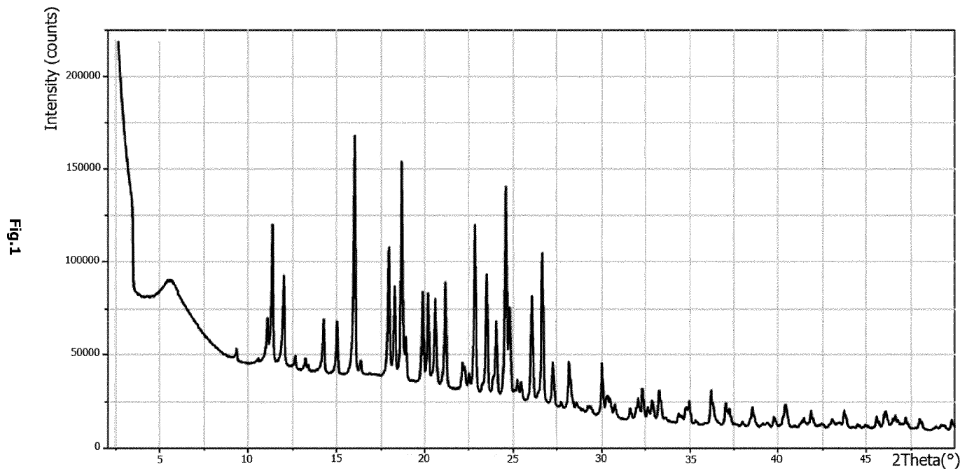

In one embodiment, the PXRD pattern of lanifibranor (beta form) is substantially in accordance with FIG. 1.

In one embodiment, lanifibranor (form beta) has a DSC curve comprising an endothermic peak at 182.3° C.

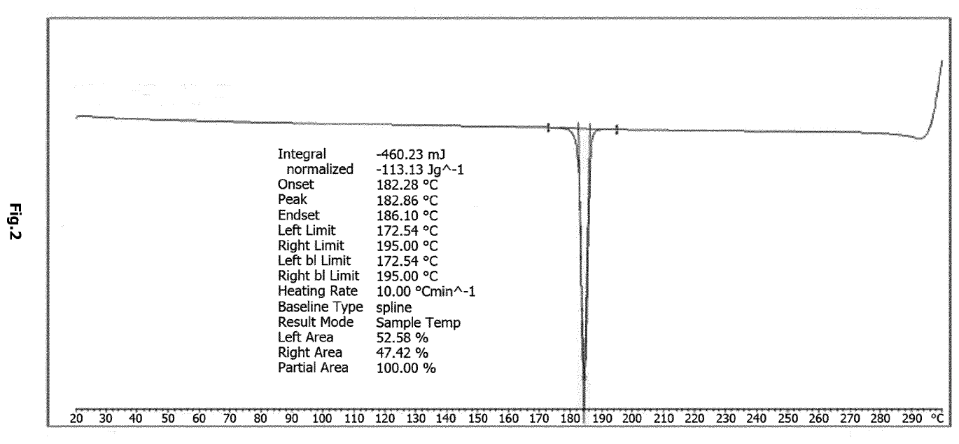

In one embodiment, the DSC curve of lanifibranor (form beta) is substantially in accordance with FIG. 2.

In one embodiment, the TGA curve of lanifibranor (form beta) does not highlight any significant weight loss over the temperature range 25-200° C. Above 250° C., the weight loss observed likely corresponds to degradation.

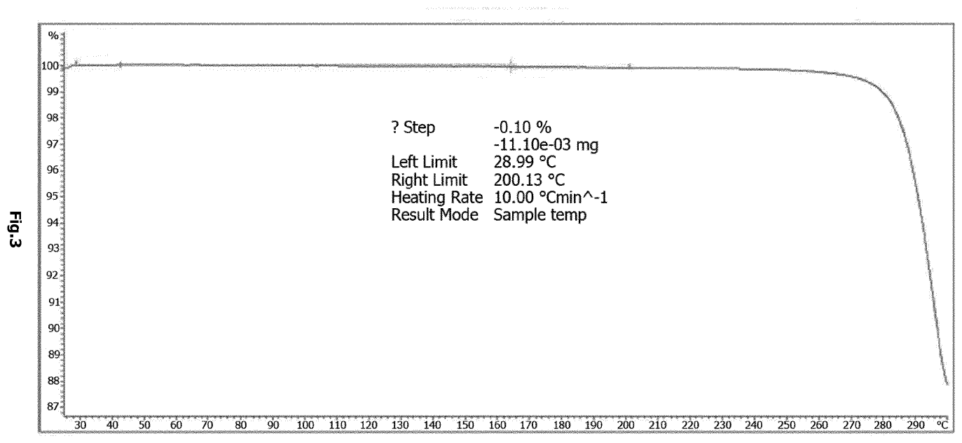

In one embodiment, the TGA curve of lanifibranor (form beta) is substantially in accordance with FIG. 3.

In one embodiment, the DVS analysis performed on lanifibranor (form beta) does not exhibit significant weight variations over the range of relative humidity values investigated: a maximum uptake of +0.1% was observed on the range 0% RH-95% RH.

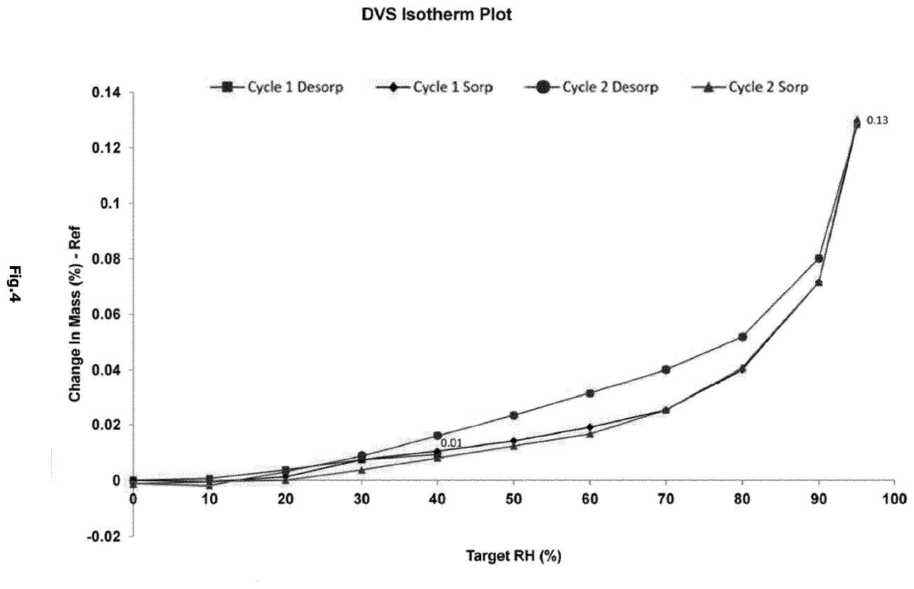

In one embodiment, the DVS isotherm plot of lanifibranor (form beta) is substantially in accordance with FIG. 4.

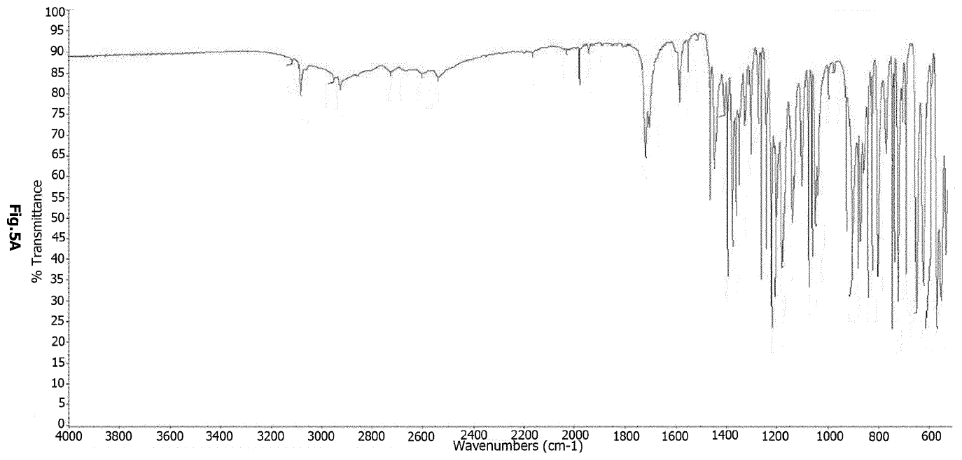

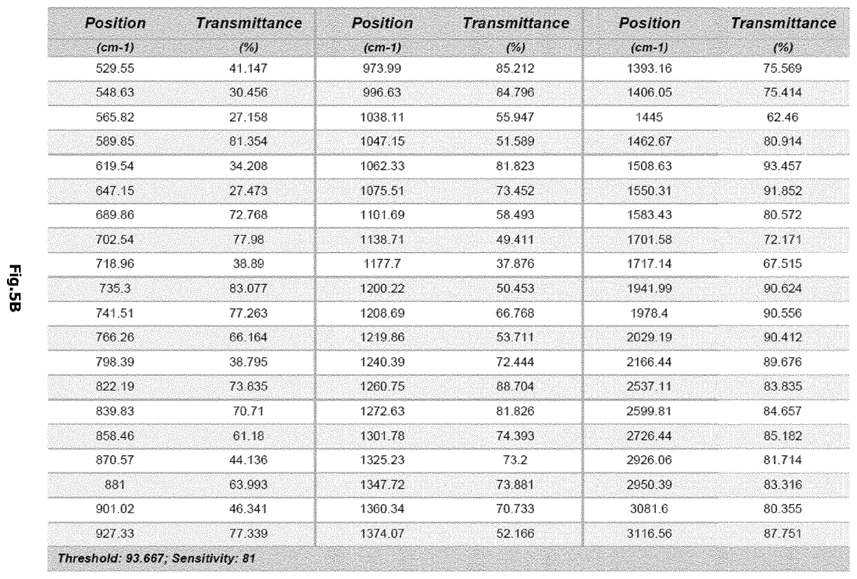

In one embodiment, the IR spectrum of lanifibranor (form beta) is substantially in accordance with FIG. 5.

In another aspect, the invention provides a method for preparing lanifibranor (form beta), the method comprising a) heating a solution of lanifibranor in acetic acid, at a temperature in the range of about 100° C. to about 110° C., and b) cooling the resulting solution to room temperature.

In one embodiment, step a) is performed at a temperature of about 105° C.

In one embodiment, step a) and step b), as defined above, are repeated at least once. For example the method for preparing lanifibranor (form beta) may comprise the steps of:
heating a solution of lanifibranor in acetic acid, at a temperature in the range of about 100° C. to about 110° C.,
allowing the solution to return to room temperature,
heating a solution of product (as recovered from the cooled solution) in acetic acid, at a temperature in the range of about 100° C. to about 110° C.,
cooling the solution to room temperature.

Lanifibranor free acid can be obtained e.g. as described in WO 2007/026097 or as described in example 1 below.

When screening for solid forms of lanifibranor, the inventors identified two crystalline forms of the compound, one form being unstable in the sense that it did readily convert to the other form upon handling/processing. The unstable form was labelled “form alpha” and the more stable form was labelled “form beta”. An amorphous form was further obtained. The amorphous form was found to crystallize into form alpha or form beta even at low temperatures. It was further found that the process for preparing form beta, where acetic acid is used for crystallization, makes it possible to scale up the manufacture of this crystalline form, to obtain batches of up to about 150 kg.

Lanifibranor form beta can be used as the active ingredient of a pharmaceutical composition. Thus, in another aspect, the invention provides a pharmaceutical composition comprising lanifibranor (form beta) and a pharmaceutically acceptable carrier or excipient. The expression “pharmaceutically acceptable carrier or excipient” means that the excipient or carrier is suitable for incorporation into a pharmaceutical composition and is compatible with the other ingredients of the composition. Particularly, it is not toxic. Its use allows facilitating the preparation, preservation and administration of the active ingredient. Such excipients and carriers are well-known to the person skilled in the art, and are described in the French and/or European Pharmacopoeia. Examples of pharmaceutical carriers include, but not limited to, any suitable solvents, dispersion media, coatings, antibacterial and antifungal agents and isotonic agents, and examples of excipients that may also be components of the formulation include fillers, binders, disintegrating agents and lubricants.

In another aspect the invention provides a method of treating non-alcoholic fatty liver disease (NAFLD), which comprises administering to a subject in need thereof an effective amount of lanifibranor (form beta).

In one embodiment, NAFLD includes non-alcoholic fatty liver (NAFL) and non-alcoholic steatohepatitis (NASH).

In another aspect the invention provides a method of treating a cirrhotic subject at risk of progressing from compensated stage to decompensated stage, which comprises administering to the subject an effective amount of lanifibranor (form beta).

In another aspect the invention provides lanifibranor form beta for use in a method of treating non-alcoholic fatty liver disease.

In another aspect the invention provides lanifibranor form beta for use in a method of treating a cirrhotic subject at risk of progressing from compensated stage to decompensated stage.

In another aspect the invention provides for the use of lanifibranor form beta as defined above for the preparation of a medicament intended for the treatment of non-alcoholic fatty liver disease (NAFLD). In some embodiments, NAFLD includes non-alcoholic fatty liver and non-alcoholic steatohepatitis.

In another aspect the invention provides for the use of lanifibranor form beta as defined above for the preparation of a medicament intended for the treatment of cirrhosis in compensated stage, especially for preventing the decompensation of cirrhosis. The invention is illustrated by the examples below.

### EXAMPLES

### Abbreviations

MeTHF=2-methyl tetrahydrofuran
MTBE=methyl tert-butyl ether
NAC=N-acetyl-L-cysteine
PdCl₂(PPh₃)₂=bis(triphenylphosphine) palladium (II) dichloride
THF=tetrahydrofuran

### PXRD

Powder X-ray diffraction analysis was conducted using a Panalytical Empyrean S3 diffractometer equipped with a Cu radiation source (Cu wavelength=1.5406 Å). Analyses were performed in transmission mode (samples were placed between Kapton® and polypropylene foils), on the angular range 20=2-50°, with a step size of 0.026° and a time per step of 20.4 s.

### DSC

DSC analysis was conducted on a Mettler Toledo DSC3+ calorimeter. Analyses were performed on a few milligrams of sample, in 40 μL sealed aluminum pans, punctured before analysis, under nitrogen flush at 50 mL/min. A temperature range between 20° C. and 300° C. was scanned at a 10° C./min rate.

### TGA

TGA analysis was conducted on a Mettler Toledo TGA/DSC3+ thermogravimetric analyzer. Analyses were performed on a few milligrams of sample, in 100 UL sealed aluminum pans, punctured before analysis, under nitrogen flush at 50 mL/min. A temperature range between 25° C. and 300° C. was scanned at a 10° C./min rate.

### DVS

DVS analysis was conducted on a SMS DVS Intrinsic system. Analyses were performed on a few milligrams of sample, in open aluminum pans at 25° C. The stability criterion for each step was a weight change lower than 0.002% on a 5 min time frame. The time criterion for each step was 100 min (minimum duration by step: 10 min). Relative humidity was scanned between 0% RH and 95% RH with 10% RH steps (40-0-95-0-95).

### IR

IR analysis was conducted on a Nicolet™ iS5 spectrometer equipped with an ATR iD7 accessory. Analyses were performed in ATR mode from 4000 cm-1 to 525 cm-1 (resolution of 4 cm-1), with 32 scans for background and measurement.

### Example 1: Lanifibranor Free Acid

Degassed triethylamine (200 mL) was added to a solution of N-(4-chloro-2-iodo-phenyl)-1,3-benzothiazole-6-sulfonamide in THF, followed by the addition of PdCl₂ (PPh3) 2 (1.17 g), CuI (0.85 g) and 5-hexynoic acid (CAS [53293-00-8]) (14.3 g). The reaction mixture was stirred at 40° C. for 3 hours. The mixture was concentrated under reduced pressure at 40° C. until 5 volumes of solution remained relative to the weight of the sulphonamide starting material. Water (3 volumes) was added, and the residual THF was removed by distillation. The mixture was washed with MTBE (2 volumes) 3 times. The aqueous phase was treated with a mixture of MeTHF and HCl (37%). The MeTHE solution was washed with a 6% aqueous solution of NAC (twice). The washing was performed by stirring the organic phase with 20% w/w of NAC at 60° C. for 1 hour. The organic phase was then washed twice and the NAC phases were combined and washed with pure water (three times). As previously, the washing was performed by stirring the NAC phase with water at 60° C. for 1 hour. The MeTHF solution was heated at 60° C. Shirasagi A Charcoal (0.5%) was added (w/w relative to the quantity of starting sulfonamide) and the suspension was stirred at 60° C. for 30 minutes. The mixture was then filtered on a dicalite bed and washed abundantly with MeTHF. The lanifibranor-containing solution obtained was used as such in the next example.

### Example 2: Lanifibranor (Form Beta)

The solution obtained in example 1 was concentrated. Acetic acid was added to the residue and the solution was concentrated. The suspension was heated at 105° C., and acetic acid was added in small portions until complete dissolution. The solution was then stirred at 105° C. for 1 hour, and left to slowly cool down to room temperature under stirring. The solid product obtained was filtered on a sintered glass G4 filter and washed twice with acetic acid to give 41.63 g of product (86.3% yield) with a HPLC purity of 99.8%. 41.3 g of product were suspended in acetic acid and the suspension was stirred at 105° C. for 1 hour. After complete dissolution, the mixture was left to cool down to room temperature in stages, filtered on a sintered glass G4 filter, and washed with acetic acid. The product collected was dried under vacuum at 60° C. for 5 hours to afford 38.6 g (93.5% yield) of the title compound.

APCI MS m/z 435 [M+H]+; UPLC-MS (210-260 nm) purity: 99.9%.

¹H NMR (500 MHZ DMSO-d6): δ 1.86-2.01 (2H, quint, J=7.5 Hz); 2.36 (2H, t, J=7.5 Hz); 3.09 (2H, t, J=7.5 Hz); 6.62 (1H, s); 7.32 (1H, dd, J=2.0 Hz et J=8.5 Hz); 7.57 (1H, d, J=2.5 Hz); 7.85 (1H, dd, J=2.0 et J=8.5 Hz); 8.09 (1H, d, J=8.5 Hz); 8.20 (1H, d, J=8.5 Hz); 8.97 (1H, d, J=2.0 Hz); 9.67 (1H, s); 12.14 (1H, s).

Form beta of lanifibranor was characterized by PXRD (FIG. 1), DSC (FIG. 2), TGA (FIG. 3), DVS (FIG. 4) and IR (FIGS. 5A and 5B). It can be seen from FIG. 2 that lanifibranor (form beta) has a DSC curve comprising an endothermic peak at 182.3° C.

### Example 3: Lanifibranor (Form Alpha)

A homogenous solution was prepared by dissolving form beta of lanifibranor (10g), as obtained in example 2, in acetic acid (7.5 volumes) at 98° C. The solution was then quenched by stirring the mixture at 5° C. under precipitation occurred. The temperature of the mixture was monitored and, once it reached 20° C., the solid phase was recovered by filtration on n°3 sintered glass filter. The solid recovered was dried at 60° C. under vacuum and kept at room temperature.

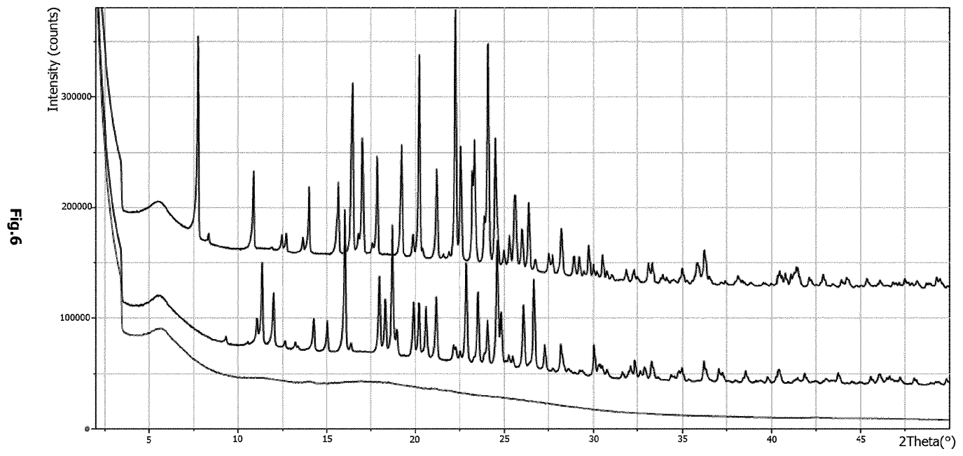

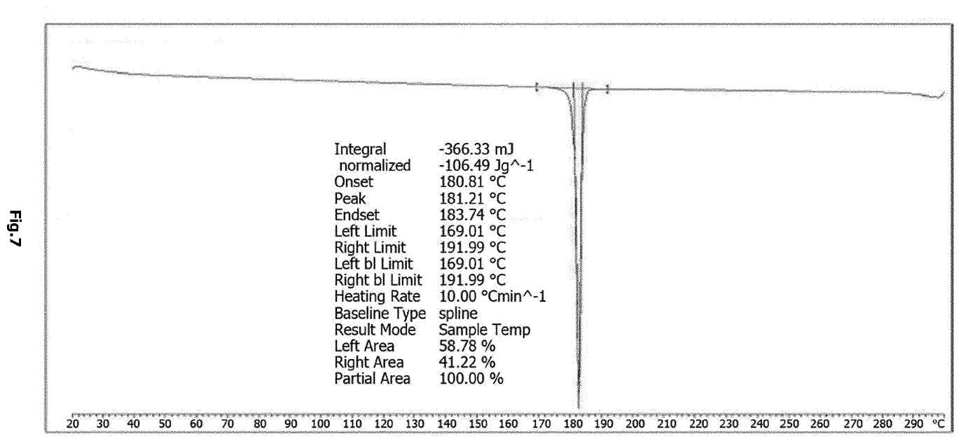

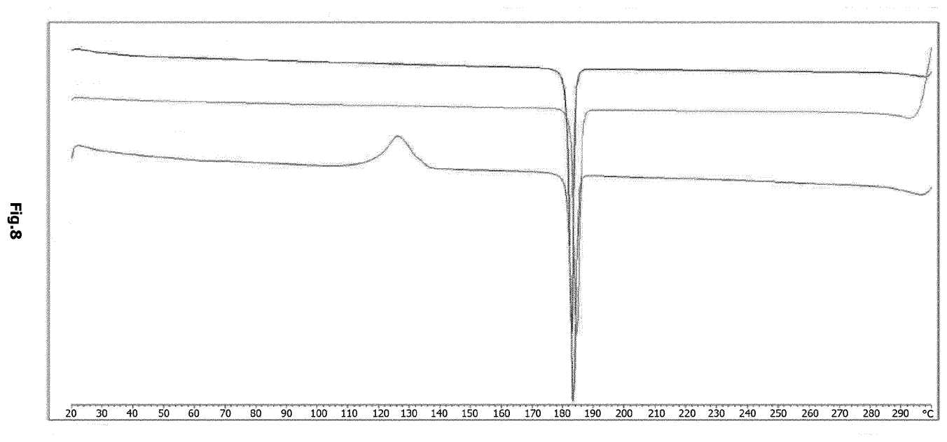

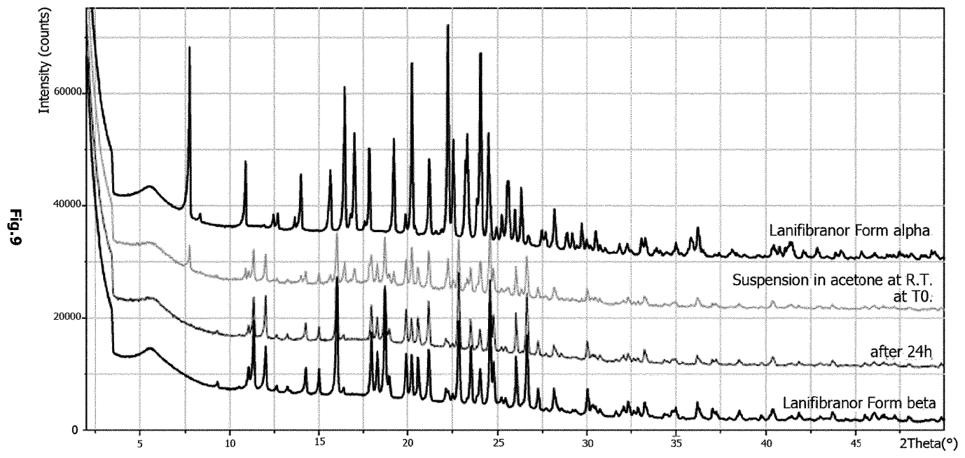

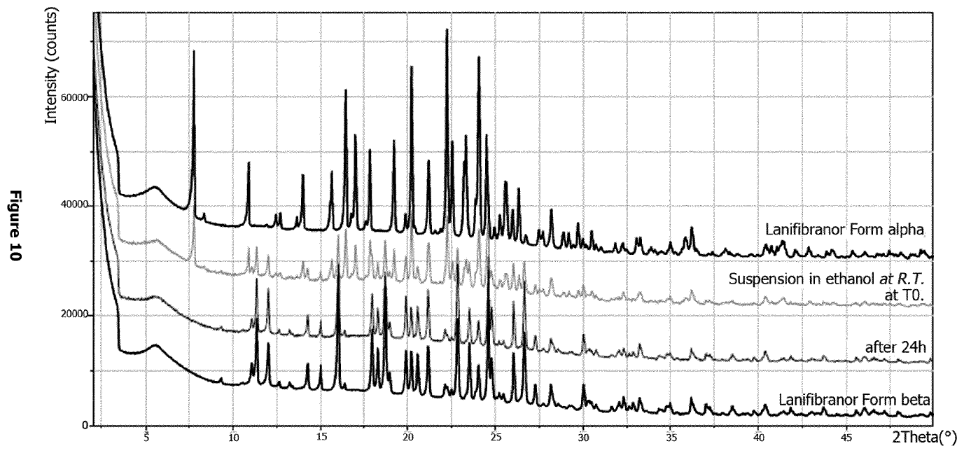

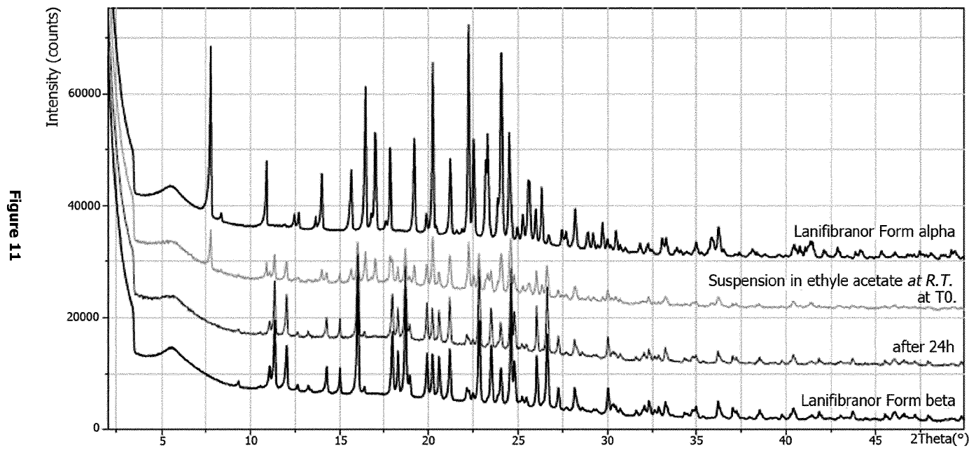

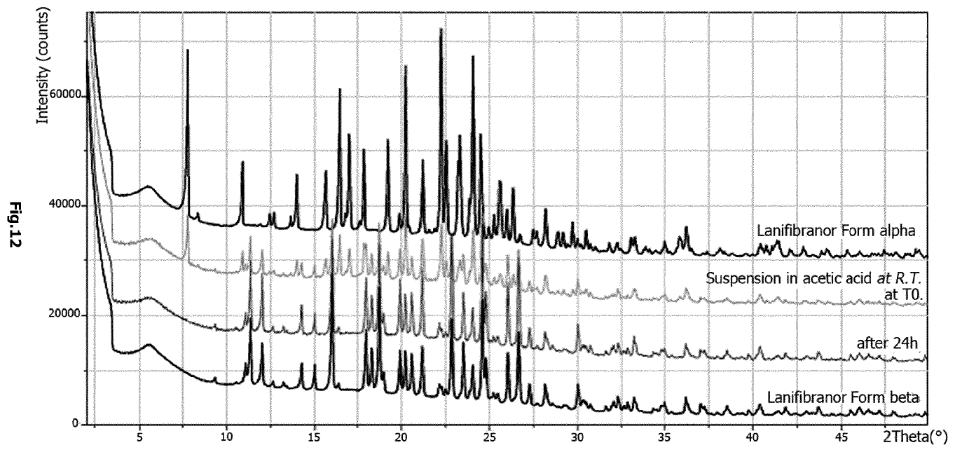

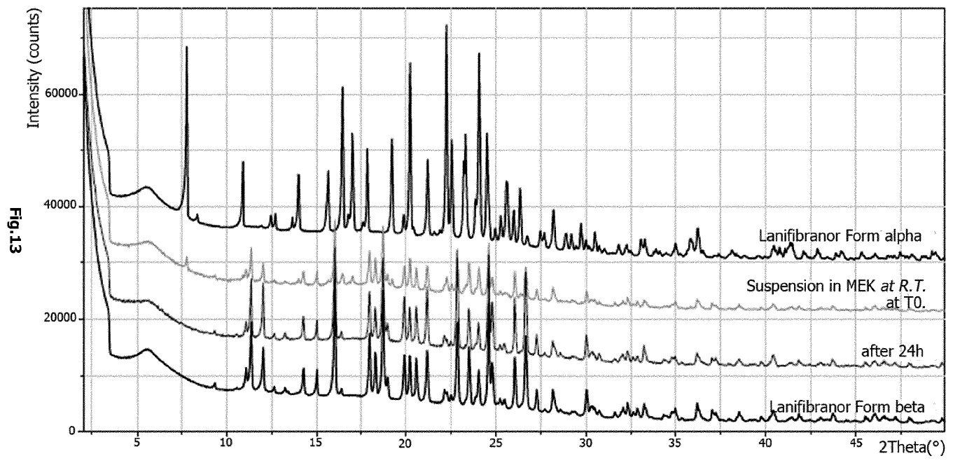

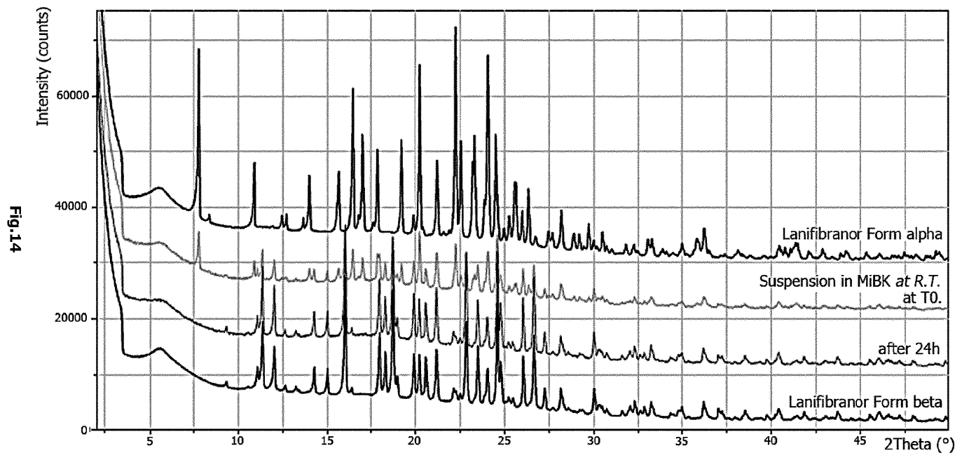

Form alpha of lanifibranor was characterized by PXRD (Table 2 and FIG. 6) and DSC (FIG. 7). It can be seen from FIG. 7 that form alpha has a DSC curve comprising an endothermic peak at 180.8° C.

TABLE 2

| 2θ position | Relative |
|---|---|
| (°) | intensity |
| 7.8 | 79.6% |
| 8.4 | 4.7% |
| 10.9 | 37.7% |
| 11.9 | 1.9% |
| 12.5 | 5.9% |
| 12.7 | 7.1% |
| 13.7 | 5.8% |
| 14.0 | 27.1% |
| 15.6 | 28.3% |
| 16.5 | 71.0% |
| 16.8 | 10.9% |
| 17.0 | 52.8% |
| 17.6 | 5.9% |
| 17.9 | 53.0% |
| 19.2 | 44.9% |
| 19.9 | 9.8% |
| 20.2 | 89.6% |
| 20.4 | 4.7% |
| 21.2 | 39.4% |
| 21.6 | 1.6% |
| 21.9 | 3.0% |
| 22.3 | 100.0% |
| 22.5 | 29.6% |
| 23.2 | 28.9% |
| 23.3 | 35.8% |
| 23.9 | 20.8% |
| 24.1 | 72.1% |
| 24.5 | 56.8% |
| 25.0 | 6.0% |
| 25.3 | 14.1% |
| 25.6 | 28.3% |
| 26.0 | 14.1% |
| 26.4 | 23.0% |
| 26.8 | 4.8% |
| 27.5 | 8.1% |
| 27.7 | 8.1% |
| 28.2 | 18.8% |
| 28.9 | 7.6% |
| 29.2 | 6.9% |
| 29.5 | 1.4% |
| 29.8 | 11.5% |
| 30.0 | 4.1% |
| 30.2 | 2.2% |
| 30.5 | 9.1% |
| 30.8 | 3.9% |
| 31.1 | 1.9% |
| 31.9 | 4.1% |
| 32.3 | 5.5% |
| 32.4 | 2.2% |
| 33.1 | 8.6% |
| 33.3 | 8.6% |
| 33.8 | 2.5% |
| 33.9 | 3.4% |
| 34.1 | 2.8% |
| 34.5 | 1.3% |
| 34.9 | 2.5% |
| 35.0 | 4.2% |
| 35.7 | 3.4% |
| 35.9 | 8.4% |
| 36.3 | 12.6% |
| 36.5 | 2.3% |
| 37.2 | 1.1% |
| 37.4 | 1.7% |
| 38.1 | 2.3% |
| 38.2 | 4.1% |
| 38.4 | 2.2% |
| 38.5 | 1.5% |

### Example 4: Lanifibranor (Amorphous Form)

A homogenous solution was prepared by dissolving 1 g of lanifibranor (form beta), as obtained in example 2, in 35 mL of acetone, at 50° C. The solution was then fully evaporated under vacuum at 50° C. The solid phase was then recovered, dried overnight under vacuum, at room temperature, and stored at −26° C.

The amorphous form of lanifibranor was characterized by PXRD (see FIG. 6).

### Example 5: Stability Tests

Competitive slurry tests were performed in various solvents such as acetone, ethanol, ethyl acetate, acetic acid, methyl ethyl ketone and methyl isobutyl ketone.

A saturated solution of lanifibranor form beta (as obtained in example 2) was prepared for each solvent. An equal amount of lanifibranor form alpha and lanifibranor form beta was introduced into a vial (50 mg+50 mg), and 250 μL of the saturated solution prepared beforehand (recovered by filtration through a 0.2 μm H-PTFE filter) were then added. The mixture was stirred at room temperature, samples of the solid were taken immediately after stirring was initiated and 24 hours after, and analyzed by PXRD to identify the solid form(s) present. The results are presented in Table 2.

TABLE 2

| | Form(s) present | Form(s) present |
|---|---|---|
| Solvent | T0 | T0 + 24 h |
| Acetone | form alpha + form beta | form beta |
| Ethanol | form alpha + form beta | form beta |
| Ethyl acetate | form alpha + form beta | form beta |
| Acetic acid | form alpha + form beta | form beta |
| Methyl ethyl | form alpha + form beta | form beta |
| ketone | | |
| Methyl isobutyl | form alpha + form beta | form beta |
| ketone | | |

It can be seen that after 24 h a total conversion towards lanifibranor form beta is observed in all solvents considered. Form beta is therefore the most stable form at room temperature.

### Example 7: Compression Tests

Compression tests were performed on the crystalline forms (alpha, beta) and on the amorphous form of lanifibranor.

100 mg of each sample were introduced between anvils into a 13 mm die and subjected to a 10-Ton pressure for 15 minutes. The pellet obtained was then de-compacted and analysed by PXRD in order to monitor changes in the crystal structure of the sample.

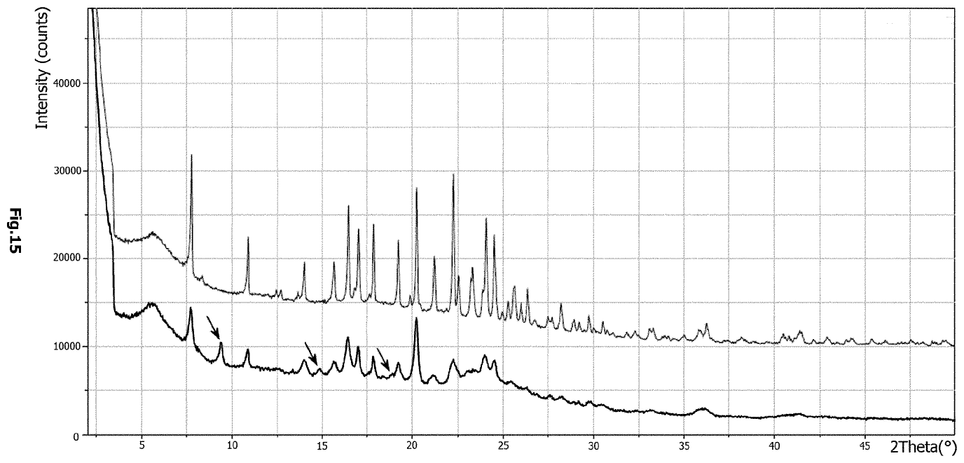

For lanifibranor form alpha (see FIG. 15), an enlargement of the diffraction peaks, a decrease of their intensities and additional signals that could correspond to peak shifts caused by a transformation to form beta were observed after compression. A signal characteristic of amorphous material was also highlighted.

For lanifibranor form beta (diffraction pattern not shown), a decrease of the crystallinity was observed after compression (peaks enlargement, decrease of intensity), but no significant change in the solid form was observed.

For the amorphous form (diffraction pattern not shown), weak diffraction signals corresponding to form beta were detected after compression, indicating the start of crystallization in the sample.

### Example 8: Comparison Between Lanifibranor Form Beta and Lanifibranor as Obtained in Example 117 of WO 2007/026097

Lanifibranor was prepared following the procedure described in example 117 of WO 2007/026097. A white amorphous powder was obtained in about 75% yield, with a melting point (measured on a Kofler bench) in the range 74-76° C. The product obtained was found to be amorphous, with a PXRD pattern comparable to that shown in FIG. 6.

### Example 9: Comparison Between Lanifibranor Form Beta and the Solid Forms Disclosed in WO 2022/122014

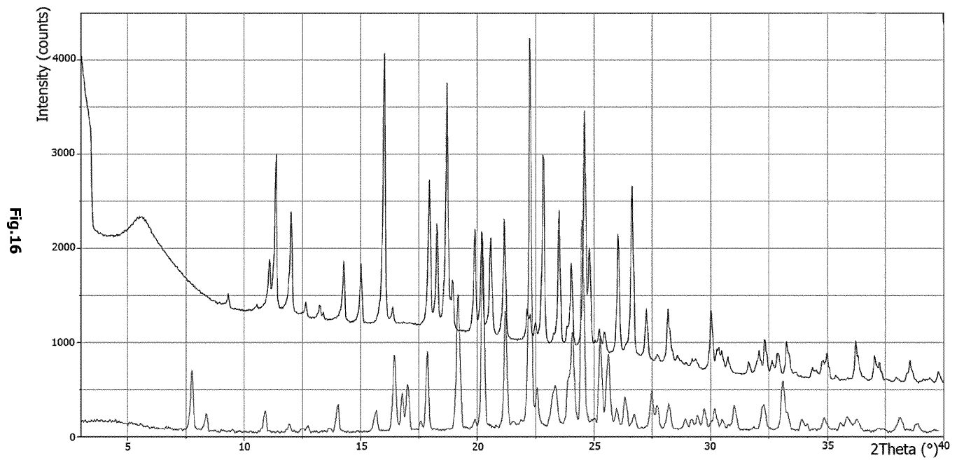

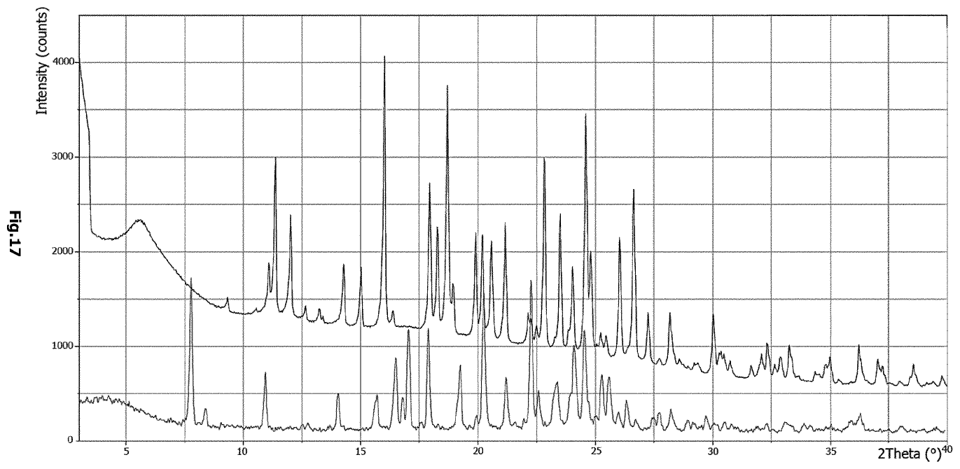

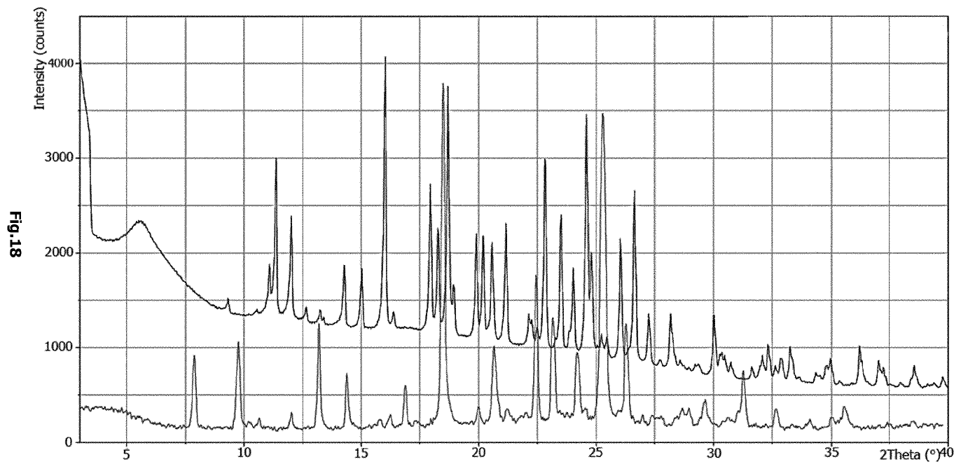

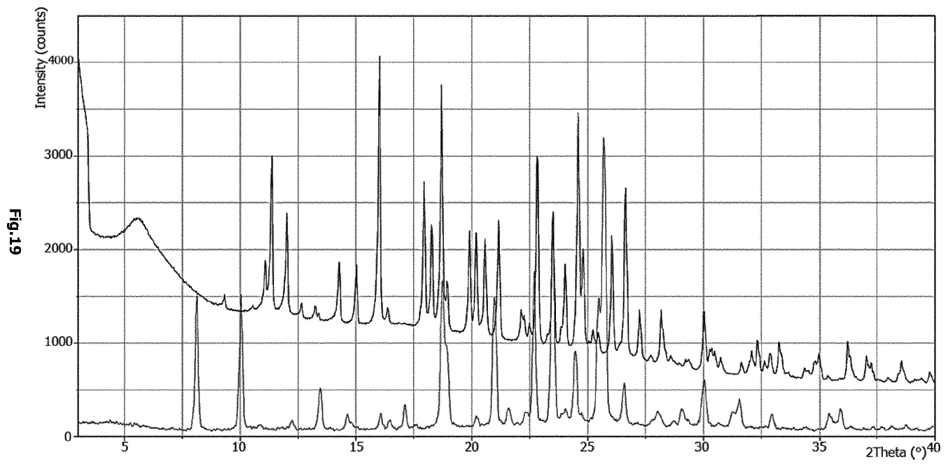

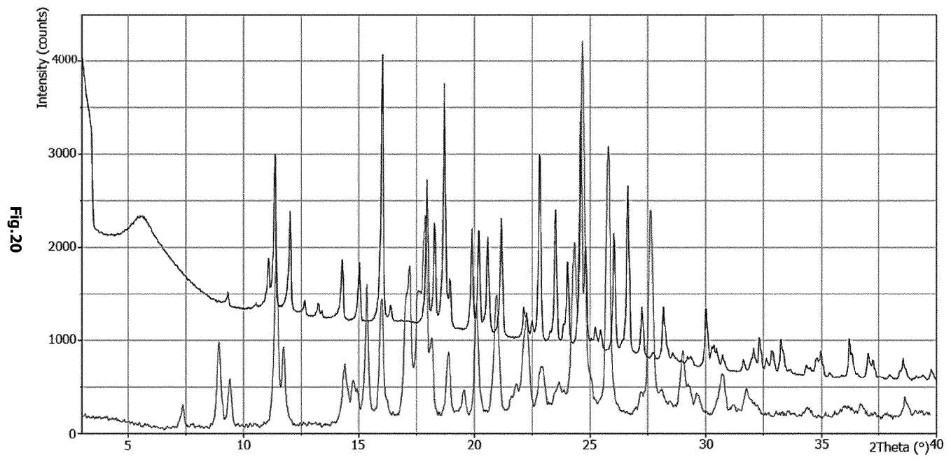

The PXRD patterns of the solid forms described in WO 2022/122014, namely form CSI (examples 2 and 3), form CSII (examples 5 and 6), and form CSIV (example 7), were digitized and then compared (by superimposition) to the PXRD pattern of lanifibranor form beta (as shown in FIG. 1). The results are shown in FIGS. 16-17 (CSI), 18-19 (CSII) and 20 (CSIV). It can be concluded from these figures that none of the PXRD patterns described in WO 2022/122014 match with the PXRD pattern of lanifibranor form beta.

### Example 10: Comparison Between Lanifibranor Form Beta and the Solid Forms Disclosed in WO 2022/143479

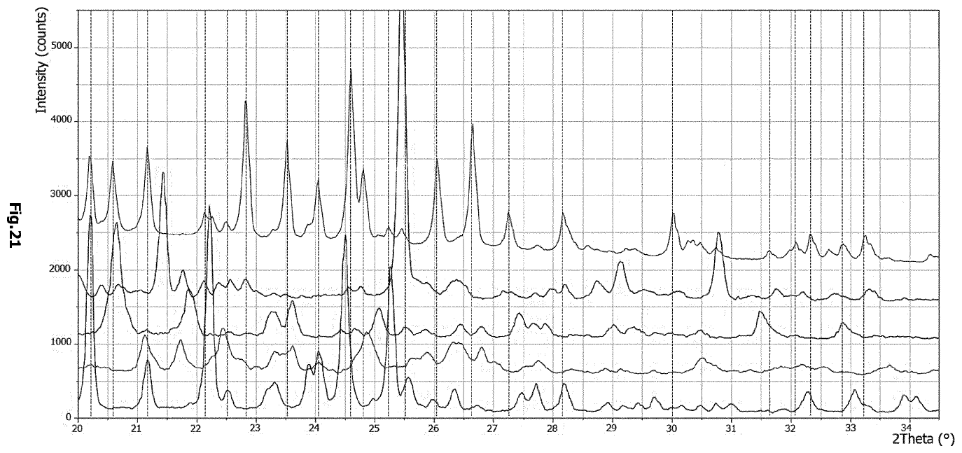

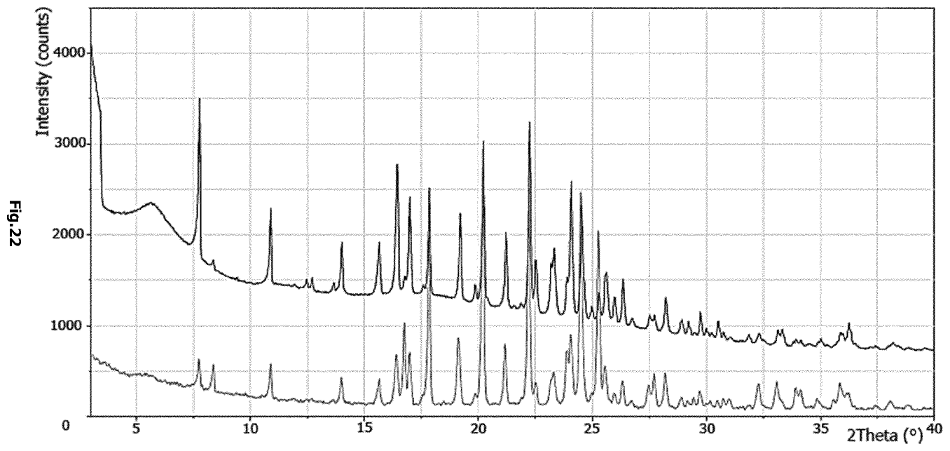

The PXRD patterns of the solid forms described in WO 2022/143479, namely lanifibranor cinnamamide co-crystal (examples 1-6), lanifibranor p-toluenesulfonic acid co-crystal (examples 7-11), lanifibranor tromethamine salt (examples 12-16), and form A (examples 17-20), were digitized and then compared (by superimposition) to the PXRD pattern of lanifibranor form beta (as shown in FIG. 1). The results are shown in FIG. 21. It can be concluded from this figure that none of the PXRD patterns described in WO 2022/143479 match with the PXRD pattern of lanifibranor form beta. By contrast, and as shown in FIG. 22, the PXRD pattern of form A described in WO 2022/143479 substantially matches with the PXRD pattern of lanifibranor form alpha (as shown in FIG. 6).

### Example 11: Comparison Between Lanifibranor Form Beta and the Solid Forms Disclosed in WO 2022/258060

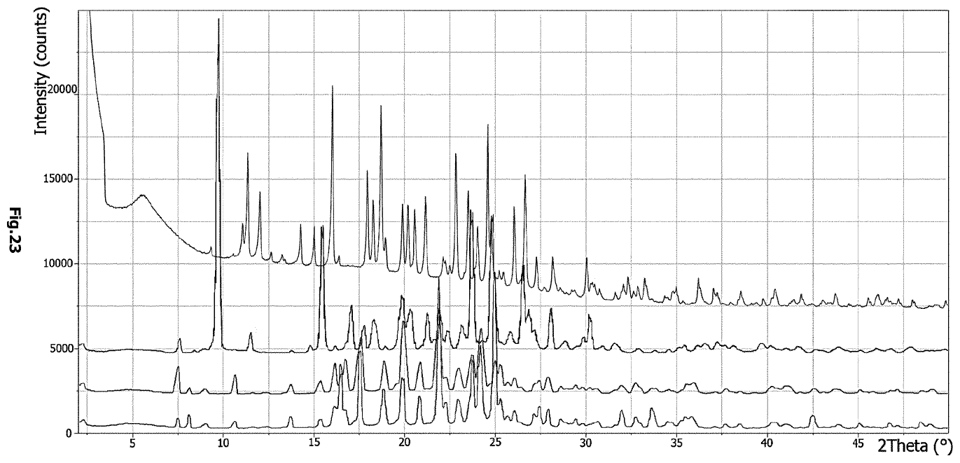

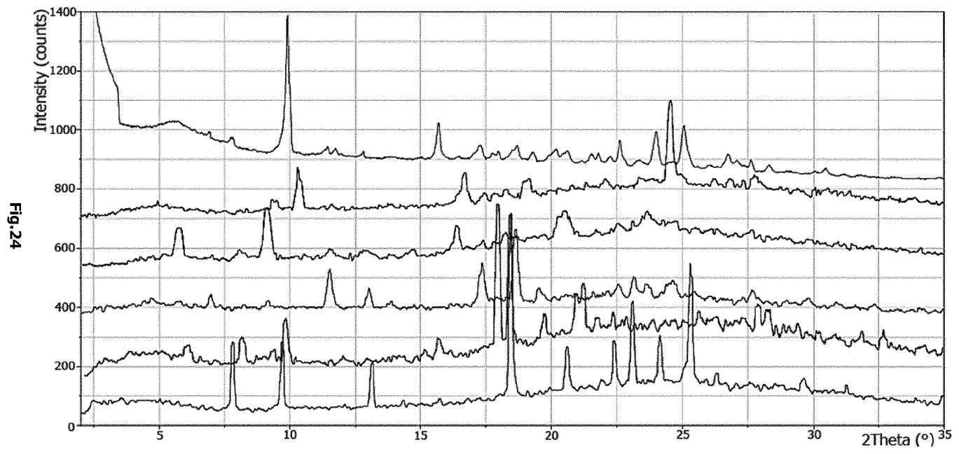

The PXRD patterns of the solid forms described in WO 2022/258060, namely lanifibranor solid forms CM-A, CM-B, CM-C, CM-D, CM-E, CM-F, CM-G and CM-I, were digitized and then compared (by superimposition) to the PXRD pattern of lanifibranor form beta (as shown in FIG. 1). The results are shown in FIG. 23 (forms CM-A, CM-B and CM-F) and FIG. 24 (forms CM-C, CM-D, CM-E, CM-G and CM-I). It can be concluded from these figures that none of the PXRD patterns described in WO 2022/258060 match with the PXRD pattern of lanifibranor form beta. In addition, the PXRD patterns of the solid forms described in WO 2022/258060 do not match either with the PXRD pattern of lanifibranor form alpha (data not shown).

### Example 12: Comparison Between Lanifibranor Form Beta and the Solid Forms Disclosed in WO 2022/261410

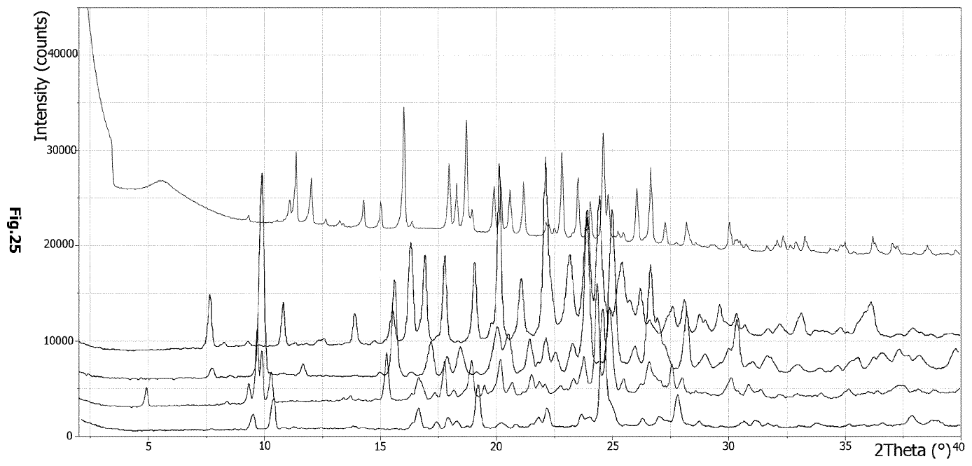

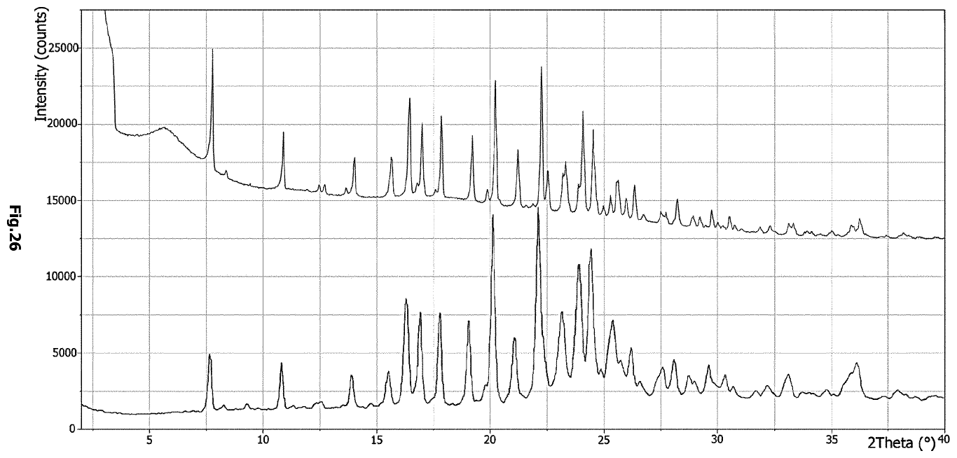

The PXRD patterns of the solid forms described in WO 2022/261410, namely lanifibranor forms LN-1, LN-2, LN-3 and LN-4, were digitized and then compared (by superimposition) to the PXRD pattern of lanifibranor form beta (as shown in FIG. 1). The results are shown in FIG. 25. It can be concluded from this figure that none of the PXRD patterns described in WO 2022/261410 match with the PXRD pattern of lanifibranor form beta. By contrast, and as shown in FIG. 26, the PXRD pattern of form LN-1 described in WO 2022/261410 substantially matches with the PXRD pattern of lanifibranor form alpha (as shown in FIG. 6).

### Example 13: Comparison Between Lanifibranor Form Beta and the Solid Forms Disclosed in WO 2023/016319

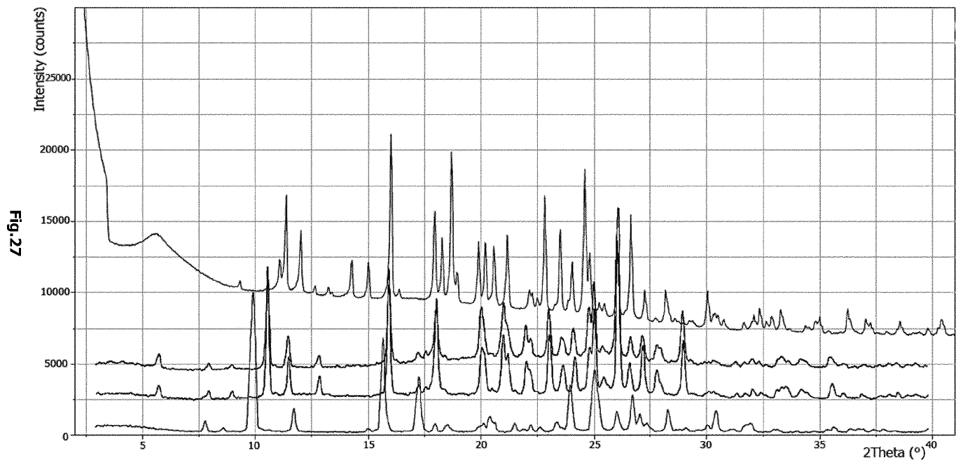

The PXRD patterns of the solid forms described in WO 2023/016319, namely lanifibranor forms CSV (embodiments 2 and 3) and CSIII, were digitized and then compared (by superimposition) to the PXRD pattern of lanifibranor form beta (as shown in FIG. 1). The results are shown in FIG. 27. It can be concluded from this figure that none of the PXRD patterns described in WO 2023/016319 match with the PXRD pattern of lanifibranor form beta. In addition, the PXRD patterns of the solid forms described in WO 2023/016319 do not match either with the PXRD pattern of lanifibranor form alpha (data not shown).

### Example 14: Stability Test

Lanifibranor form beta, as obtained in example 2, was submitted to stability testing in accordance with EMA guidance (CPMP/ICH/2736/99, last revised in August 2003); the drug substance was thus stored under the following conditions:
6 months at 30° C./65° RH,
6 months at 40° C./75% RH, and
12 months at 25° C./60% RH.

At the end of each storage period, the drug substance was characterized by PXRD and by DSC. In each case, the PXRD pattern was characteristic of lanifibranor form beta as shown in FIG. 1, and the DSC curve was characteristic of lanifibranor form beta (endothermic peak at 182.3° C.), indicating that lanifibranor form beta is stable over long-term storage.

1-15. (canceled)

16. A crystalline form of lanifibranor having an X-ray powder diffraction pattern comprising peaks at 11.1°+0.2°, 12.0°+0.2°, and 15.0°+0.2° 20, as measured using an X-ray wavelength of 1.5406 Å.

17. The crystalline form of lanifibranor of claim 16, further comprising a peak at 20.6°+0.2° 20, as measured using an X-ray wavelength of 1.5406 Å.

18. The crystalline form of lanifibranor of claim 16, further comprising a peak at 26.7°+0.2° 20, as measured using an X-ray wavelength of 1.5406 Å.

19. The crystalline form of lanifibranor of claim 16, further comprising at least one peak at 20 values selected from 16.0°+0.2°, 18.7°+0.2° and 24.6°+0.2° 20, as measured using an X-ray wavelength of 1.5406 Å.

20. The crystalline form of lanifibranor of claim 16, further comprising at least one peak at 20 values selected from 21.2+0.2° and 26.1+0.2° 20, as measured using an X-ray wavelength of 1.5406 Å.

21. The crystalline form of lanifibranor of claim 16, further comprising at least one peak at 20 values selected from 11.4°+0.2°, 18.0°+0.2°, 22.8°+0.2° and 23.5°+0.2°, as measured using an X-ray wavelength of 1.5406 Å.

22. A crystalline form of lanifibranor having an X-ray powder diffraction pattern comprising peaks at 15.0°+0.2°, 16.0°+0.2°, 18.7°+0.2°, and 24.6°+0.2° 20, as measured using an X-ray wavelength of 1.5406 Å.

23. The crystalline form of lanifibranor of claim 22, further comprising a peak at 20.6°+0.2° 20, as measured using an X-ray wavelength of 1.5406 Å.

24. The crystalline form of lanifibranor of claim 22, further comprising a peak at 26.7°+0.2° 20, as measured using an X-ray wavelength of 1.5406 Å.

25. The crystalline form of lanifibranor of claim 22, further comprising peaks at 11.1°+0.2° and 12.0°+0.2° 20, as measured using an X-ray wavelength of 1.5406 Å.

26. The crystalline form of lanifibranor of claim 16, wherein the X-ray diffraction pattern is substantially in accordance with FIG. 1.

27. The crystalline form of lanifibranor of claim 16, having a differential scanning calorimetry (DSC) curve comprising an endothermic peak at 182.3° C.

28. The crystalline form of lanifibranor of claim 27, wherein the DSC curve is substantially in accordance with FIG. 2.

29. A pharmaceutical composition comprising the crystalline form of lanifibranor of claim 16 and a pharmaceutically acceptable carrier or excipient.

30. A pharmaceutical composition comprising the crystalline form of lanifibranor of claim 22 and a pharmaceutically acceptable carrier or excipient.

31. A process for preparing a crystalline form of lanifibranor, comprising:
(a) heating a solution of lanifibranor in acetic acid, at a temperature in the range of about 100° C. to about 110° C., and
(b) cooling the resulting solution to room temperature.

32. The process of claim 31, wherein step (a) is performed at a temperature of about 105° C.

33. The process of claim 31, wherein step (b) is performed in stages.

34. A method of treating non-alcoholic fatty liver disease (NAFLD) in a subject, comprising administering a therapeutically effective amount of the crystalline form of lanifibranor of claim 16 to the subject.

35. The method of claim 34, wherein NAFLD includes non-alcoholic fatty liver and/or non-alcoholic steatohepatitis.

36. A method of treating liver cirrhosis in a subject, comprising administering a therapeutically effective amount of the crystalline form of lanifibranor of claim 16 to the subject.

37. The method of claim 36, wherein the subject is at risk of progressing from compensated stage to decompensated stage.

38. A method of treating non-alcoholic fatty liver disease (NAFLD) in a subject, comprising administering a therapeutically effective amount of the crystalline form of lanifibranor of claim 22 to the subject.

39. The method of claim 38, wherein NAFLD includes non-alcoholic fatty liver and/or non-alcoholic steatohepatitis.

40. A method of treating liver cirrhosis in a subject, comprising administering a therapeutically effective amount of the crystalline form of lanifibranor of claim 22 to the subject.

41. The method of claim 40, wherein the subject is at risk of progressing from compensated stage to decompensated stage.
